(19) United States
(12) Patent Application Publication
(10) Pub. No.: US 2012/0029458 A1
(43) Pub. Date: Feb. 2, 2012

(54) Bag

(76) Inventors: Joshua James Norman, Cincinnati, OH (US); Jie Wei, South Pasadena, CA (US); Donald Carroll Roe, West Chester, OH (US)

(21) Appl. No.: 13/189,766

(22) Filed: Jul. 25, 2011

Related U.S. Application Data
(60) Provisional application No. 61/367,978, filed on Jul. 27, 2010.

(51) Int. Cl. A61F 13/84; B65D 30/00

(52) U.S. Cl. 604/385.14; 383/4

(57) ABSTRACT

A bag has a first, water resistant configuration and a second, water permeable configuration. The bag may contain liquid and/or solid soils in the first configuration, and be permeable to wash liquor in the second configuration. The bag may be adapted to hold a clean article against the exterior of the bag. The bag may be rolled for storage. A method for using the bag to store and launder one or more articles and a kit comprising the bag and one or more reusable absorbent article components are described.

### CROSS REFERENCE TO RELATED APPLICATIONS

This application claims the benefit of U.S. Provisional Application No. 61/367,978, filed on Jul. 27, 2010, which is hereby incorporated by reference in its entirety.

### FIELD OF THE INVENTION

The disclosure relates to a bag useful for washing garments, and more particularly to a bag which is resistant to liquid soils in a first configuration, and permeable to wash liquid in a second configuration.

### BACKGROUND OF THE INVENTION

Absorbent articles worn on or near the body may be used to capture and store body exudates, such as urine, feces, menses, and the like. Absorbent articles may be disposable. That is, they may be intended to be worn for some period or time, or until they have been soiled with one or more body exudates, and then discarded. Absorbent articles may include reusable components. For example, an absorbent article may have a reusable outer cover. The reusable outer cover may be used, for example, with disposable inserts. The disposable insert may provide absorbency, and the reusable outer cover may comfortably hold the disposable insert in place. A reusable outer cover may also be used with a reusable absorbent insert, such as a cloth diaper. Relative to a fully disposable absorbent article, a reusable outer cover may provide environmental benefits, even when used with a disposable absorbent insert. For example, the reusable outer cover may reduce the amount of waste that will be disposed of in a landfill, relative to a disposable absorbent article.

Because the reusable outer cover is not disposed of after use, the wearer or a caregiver using a partially or fully reusable absorbent article may be confronted with the problem of storing or transporting a soiled reusable outer cover. For example, if the reusable outer cover is soiled while the wearer is away from home, it may be necessary or desirable to bring the reusable outer cover home for laundering. Users of absorbent articles may routinely carry a bag, such as a diaper bag or purse, which may be used to store, for example, fresh absorbent articles. However, it may be undesirable to place a soiled article into a diaper bag or purse, as the soiled article may contaminate clean surfaces or contents inside the diaper bag or purse.

It is, of course, possible to place soiled reusable outer covers into a separate bag and place that bag into a diaper bag or purse. This approach may still have drawbacks. For example, if disposable bags are used, the environmental benefits of using an at least partially reusable absorbent article may be reduced or entirely offset by the disposal of the bags. As another example, whether disposable or reusable bags are used, it may be necessary for the wearer or caregiver to reach into the bag and remove soiled outer covers by hand to launder the soiled covers. The prospect of reaching into a bag which may contain body fluids to remove soiled covers for laundering may discourage the use of reusable absorbent articles.

There is a need for a system for storing, transporting, and laundering soiled reusable absorbent articles which does not require handling the soiled articles more than once, risk contaminating clean surfaces, or offset the environmental benefits of using a reusable absorbent article.

### SUMMARY OF THE INVENTION

In some aspects, the invention relates to a bag. The bag may comprise a water resistant material. The bag may comprise a water permeable material joined to the water resistant material. The water resistant material and the water permeable material may be joined to form a continuous interior space. The bag may have a first configuration, wherein the water permeable material is cabined within the water resistant material. The bag may have a second configuration, wherein the water permeable material is exposed. The bag may have a first opening to access the continuous interior space. The bag may have a second opening to expose the water permeable material. The second opening may comprise a closure having an open position and a closed position. In the closed position, the closure for the second opening may hold the bag in the first configuration.

The bag may comprise a funnel between the water permeable material and the water resistant material within the continuous inner space. A one-way valve may be attached to the funnel. The continuous interior space may be resistant to liquid in the first configuration and permeable to liquid in the second configuration. The bag may comprise at least one intercompartmental fastener between the water resistant material and the water permeable material. The bag may comprise a closure to hold the bag in a rolled or folded configuration. The bag may have a first outer surface and a second outer surface opposite the first outer surface. At least one of the first outer surface and the second outer surface may comprise a connector. The connector may be for holding an absorbent article. At least one of the first outer surface and the second outer surface may comprise an absorbent material.

The water resistant material may comprise a laminate of a water permeable material and a film. The water permeable material may comprise a mesh or netting. The mesh or netting may have an average hole size of at least 2 mm², or at least 5 mm².

In other aspects, the invention relates to a kit comprising a bag and one or more reusable absorbent article components. The bag and at least one of the one or more reusable absorbent article components are configured to be reversibly joined together. The kit may comprise instructions for using the bag. The kit may comprise instructions for using the one or more reusable absorbent article components. The one or more reusable absorbent article components may include a reusable outer cover. The kit may comprise at least one disposable absorbent insert.

### BRIEF DESCRIPTION OF THE DRAWINGS

FIG. 1 is a perspective view of an exemplary bag in a first, water resistant configuration.

FIG. 2 is a perspective view of an exemplary bag in a second, water permeable configuration.

FIG. 3 is a side view of an exemplary bag in a second, water permeable configuration.

FIG. 4 is a perspective view of an exemplary bag in a first, water resistant configuration, the bag containing exemplary soiled articles.

FIG. 5 is a perspective view of an exemplary bag in a second, water permeable configuration, the bag containing exemplary soiled articles.

FIG. 6 is a schematic view of an exemplary bag having intercompartmental fasteners.

FIG. 7 is a schematic view of an exemplary bag having a funnel.

FIG. 8A is a side view of an exemplary bag having a roll closure.

FIG. 8B is a side view of an exemplary bag having a roll closure.

FIG. 9 is a perspective view of an exemplary bag having article connectors.

FIG. 10 is a perspective view of an exemplary bag partially rolled with an absorbent article.

FIGS. 11A-11D are exemplary images used in the Average Hole Size and Percent Open Area of Mesh Test.

### DETAILED DESCRIPTION OF THE INVENTION

As used herein, “water resistant” refers to a material or structure which tends not to transmit liquid from one side of the material or the inside of the structure to the other side of the material or the outside of the structure.

As used herein, “water permeable” refers to a material or structure which allows liquid to move freely from one side of the material or the inside of the structure to the other side of the material or the outside of the structure.

As used herein, “wash cycle” refers to the collective steps used to launder an article or group of articles. A wash cycle may comprise, for example, soaking or agitation in wash liquor, and one or more rinsing steps. The wash liquor may be only water, or may include additives such as detergents, surfactants, stain removal products, fabric softeners, bleach, bleach alternatives, or other products for cleaning, maintaining, restoring, or treating fabrics. Rinsing liquids, if used, may be only water, or may include products for cleaning, maintaining, restoring, or treating fabrics. The products, if any, used in the wash liquor and the rinse liquid(s) may be different. For example, the wash liquor may include a detergent, and the rinsing liquid may include a fabric softener or anti-static treatment. A wash cycle may be performed by hand or by machine.

The present disclosure relates to a bag. The bag may have a first configuration, which may be water resistant. In the first configuration, the bag may be used to store and/or transport soiled absorbent articles. Because the bag is water resistant in the first configuration, it may be helpful in carrying soiled articles without transferring soils from the soiled articles to other surfaces. The bag may have a second configuration, in which at least a portion of the bag is water permeable. In the second configuration, the bag may be used to launder soiled articles.

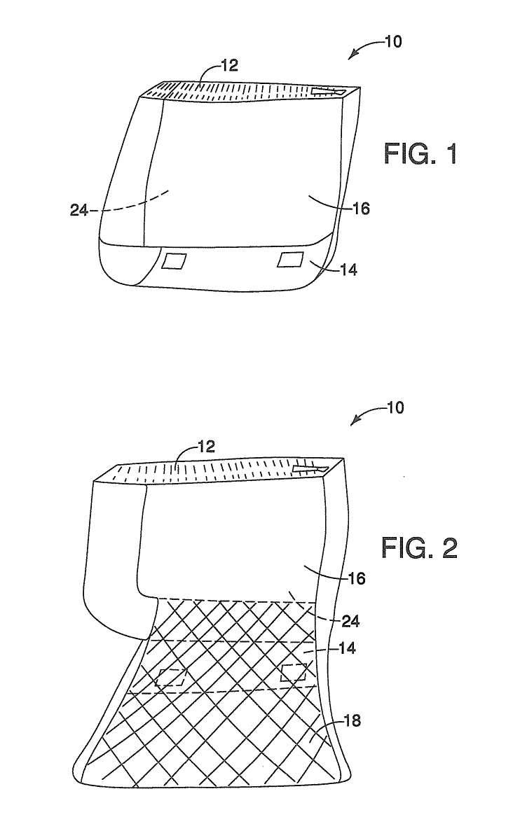

FIG. 1 shows an exemplary bag 10 comprising a first opening 12, a second opening 14, and a water resistant material 16. First opening 12 provides access to a water resistant compartment defined by water resistant material 16. Second opening 14 provides access to a water permeable compartment defined by water permeable material 18 (shown in FIG. 2). Water resistant material 16 and water permeable material 18 may be joined to form a continuous inner space 24. Water permeable material 18 may be folded, rolled, gathered, or crammed inside the water resistant compartment defined by water resistant material 16. When water permeable material 18 is cabined within water resistant material 16 and second opening 14 is closed, bag 10, including continuous inner space 24, may be water resistant. When second opening 14 is opened, water permeable material 18 is exposed, that is, water permeable material 18 may no longer be cabined within water resistant material 16, and bag 10, including continuous inner space 24, may be water permeable. Thus, bag 10 can be converted between a first, water resistant configuration to a second, water permeable configuration by manipulating second opening 14.

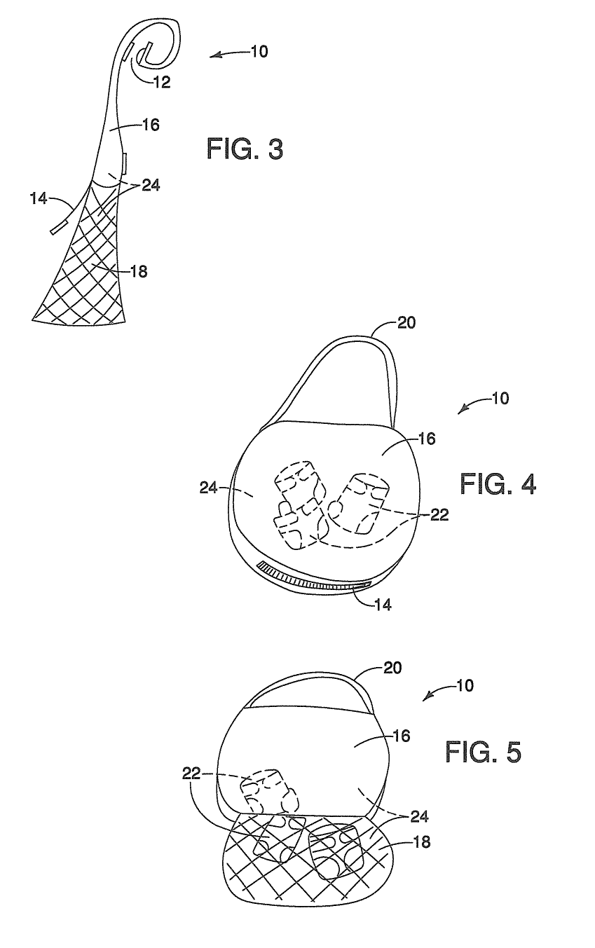

First opening 12 and second opening 14 may each comprise water resistant closures, such that soils within the water resistant compartment are unlikely to exit the bag via first opening 12 or second opening 14 when the closures are closed. First opening 12 and second opening 14 may have refastenable closures, such that each closure can be opened and closed multiple times over the life of the bag. In FIG. 1, first opening 12 is shown having a zipper and second opening 14 is shown having hook-and-loop fasteners, however, almost any fastening system may be used. For example, in addition to zippers or hook-and-loop fasteners, first opening 12, second opening 14, or both, may comprise fasteners such as drawstrings, snaps, buttons, hook-and-hook, hook-and-eye, cohesives, magnets, and the like. Fasteners, such as intermittent snaps, which might not provide a water resistant closure may be used in conjunction with folds, rolls, or other structures to limit the movement of soils from inside the bag to the area near the closure. An example of a folded or rolled closure is shown in first opening 12 in FIG. 3. In some embodiments, a pre-formed fold or roll does not require a separate fastener to hold the fold or roll in a closed position.

Water resistant material 16 may be resistant to liquid or solid soils or both. Water resistant material 16 may have a hydrohead value, using the test method described below, greater than about 10 mbar, or about 50 mbar, or about 100 mbar, or about 200 mbar, in order to contain liquid soils, such as urine or the liquid components of feces or menses, under typical use conditions. Exemplary water resistant materials include, but are not limited to, water impermeable films such as polyolefin or polyurethane films. Water resistant material 16 does not need to be inherently water resistant. For example, a water permeable material, such as cotton, may be used in combination with a water resistant polymer film, as a laminate or as separate layers. The polymer film may be placed on the inside of the bag, on the outside of the bag, or on both the inside and the outside of the bag. Polyolefin or polyurethane films may be heat laminated to a woven or non-woven fabric to create a water-resistant material. Non-water-resistant materials may be treated to confer water resistance. For example, non-water-resistant materials may be blended (as with micro or nanofibers), coated, or otherwise treated with water resistant compounds, such as polytetrafluoroethylene (also known as PTFE, or TEFLON); fluorocarbon derivatives (also known as SCOTCHGARD, as described, for example, in U.S. Pat. No. 3,574,791); or a hydrophobic silicone polymer or fluorinated polymer (such as those described, for example, in U.S. Pat. No. 7,626,073). Non-water-resistant materials may also be used if they are sufficiently dense and/or thick that the material is unlikely to permit liquid or solid soils from readily passing through the material (as opposed to leaking from seams or closures). Some woven or non-woven fabrics may have small fiber size and small spaces between the fibers, resulting in a low capillary pressure that renders the fabric water resistant. In some embodiments, water resistant material 16 is waterproof.

Water permeable material 18 may comprise any material that allows the free movement of water and wash liquor across the material. In some embodiments, water permeable material 18 is discontinuous. For example, water permeable material 18 may be a mesh or netting. If water permeable material 18 is a mesh or netting, the openings between the structural threads, cords, ropes, or strips of material forming the mesh or netting may be just large enough to render the material water permeable, or just small enough to contain the desired articles inside bag 10 when water permeable material 18 is exposed. The spaces between the structural elements may be varied, or irregular, such that some openings are of a different size, shape, or orientation than other openings. The openings may form an aesthetically pleasing pattern. For example, the openings and the structural elements between the openings may form shapes or images, such as flowers, stars, geometric patterns, and the like. The openings may have an average hole size of at least about 2 mm², or at least about 5 mm², or at least 10 mm², measured using the Average Hole Size and Percent Open Area of Mesh test described below. The openings may have an average hole size, for example, between 4 mm² and 15 mm². The percent open area may be greater than about 30%, or greater than about 50%. The percent open area may be less than about 95%.

Both water resistant material 16 and water permeable material 18 may be launderable. That is, both water resistant material 16 and water permeable material 18 may be able to withstand at least 10, or at least 50, cycles of machine washing and drying, as defined by AATCC Test Method 124-2001, with modifications as described below. Water resistant material 16, water permeable material 18, or both may be odor proof or odor resistant. That is, water resistant material 16, water permeable material 18, or both may tend not to hold or transmit odor-causing compounds or odor-causing organisms such as bacteria or fungi. Any anti-microbial treatment that can be applied to fibers or fabrics, including, but not limited to, the inclusion of silver particles in the fabric, may be used.

FIG. 4 shows an alternate exemplary bag 10 having a carrying strap 20. FIG. 4 shows three soiled articles 22 disposed in continuous inner space 24 of bag 10. Bag 10 may be sized to hold at least one, two, three, four, five, or six soiled articles 22. If soiled articles 22 are reusable outer covers for an absorbent article, sizing bag 10 to hold at least two or three soiled articles may enhance the environmental advantages of using a reusable outer cover for an absorbent article by capitalizing on the energy and water committed to a wash cycle, relative to washing only one soiled article at a time. Where bag 10 is used to launder reusable outer covers for absorbent articles, it may be unnecessary to size bag 10 to hold more than six or seven soiled articles, as some users of absorbent articles may possess only seven or eight reusable outer covers. Thus, allowing for one cover in use (on the wearer), no more than seven soiled articles might need to be laundered at one time, and a larger bag 10 may be cumbersome, or may inhibit the ability to wash other articles with bag 10, or may not be compatible with smaller wash basins. Bag 10 may be sized to accommodate one or more reusable absorbent inserts and one or more reusable outer covers at the same time. Bag 10 could be adapted for use by an institution, such as a diaper service which accepts soiled articles and returns clean articles, a daycare facility, a group home, or the like, which might process large numbers of soiled absorbent articles. For such use, bag 10 might be configured to hold 10, or 20, or 50, or 100, or even more soiled absorbent articles. FIGS. 4 and 5 show soiled articles 22, which may be reusable outer covers, cloth diapers, other reusable absorbent inserts, or a combination of these. Of course, bag 10 may also be used to handle other soiled articles, such as swimsuits, athletic wear, intimates, washable toys, and the like.

FIG. 5 shows bag 10 with water permeable material 18 exposed. Because water resistant material 16 and water permeable material 18 are joined to form continuous inner space 24, soiled articles 22 may be able to move from the area enclosed by water resistant material 16 to the area enclosed by water permeable material 18, and vice versa. Continuous inner space 24 may allow water and wash liquor to enter the area enclosed by water resistant material 16 when water permeable material 18 is exposed, that is, when bag 10 is in the second configuration. Thus, laundering bag 10 while water permeable material 18 is exposed may clean both soiled articles 22, if present, and bag 10. Both soiled articles 22 and bag 10 may be laundered by opening second opening 14, and, if desired, shaking or rotating bag 10 to shift soiled articles into the area enclosed by water permeable material 18. It might not be necessary to reach into bag 10 or remove soiled articles 22 from bag 10 while they are soiled in order to launder bag 10 or soiled articles 22. Once soiled articles 22 are placed in bag 10, it might not be necessary to directly handle soiled articles 22 until they are clean, thus reducing a user or caregiver's contact with soiled articles 22, such as urine-, feces-, or menses-contaminated articles. Because soiled articles 22 are contained in bag 10 during laundering, soiled articles 22 may be protected from damage from other articles in the wash cycle, and other articles in the wash cycle may be protected from damage from soiled articles 22. For example, soiled articles 22 may comprise hook-and-loop fasteners which, if they came into contact with other articles in the wash cycle, might cause tears, pilling, pulls, or other damage.

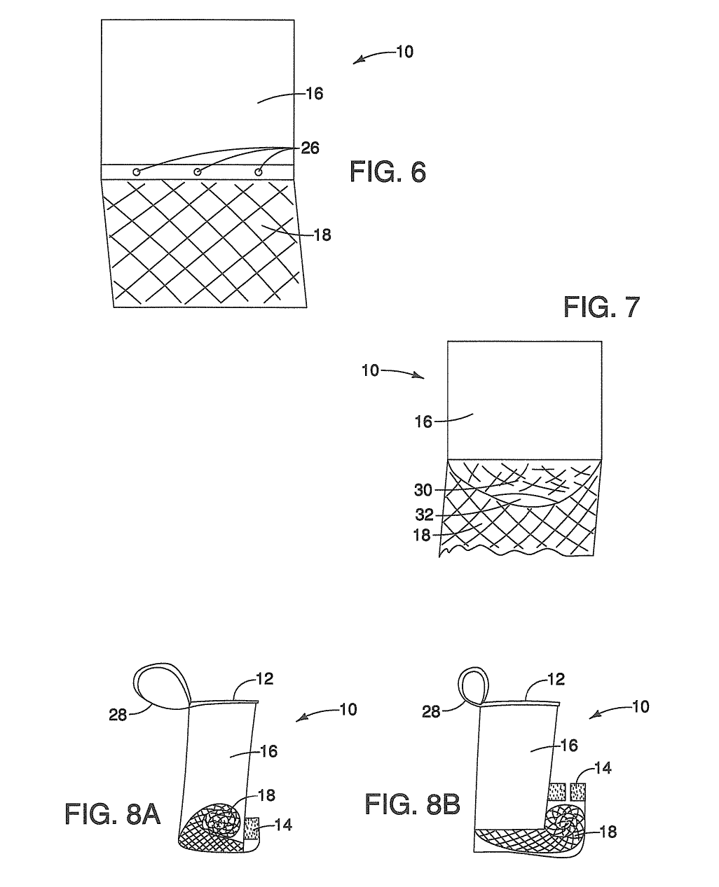

FIGS. 6-8 show exemplary bags 10 having additional, optional features. For example, FIG. 6 shows bag 10 having intercompartmental fasteners 26, shown as snaps, between the area enclosed by water resistant material 16 and the area enclosed by water permeable material 18. Intercompartmental fasteners 26 may divide the continuous interior space 24 into two compartments, a first compartment roughly defined by the area enclosed by water resistant material 16, and a second compartment roughly defined by the area enclosed by water permeable material 18. Prior to laundering, soiled articles 22 may be shaken or manipulated into the second compartment defined by water permeable material 18, and intercompartmental fasteners 26 may be engaged to prevent soiled articles 22 from moving back into the first compartment defined by water resistant material 16 during the wash cycle. If intercompartmental fasteners 26 do not create a water-resistant or water-proof seal, the inside of bag 10 may still be cleaned during the wash cycle. Such compartmentalization may be helpful in exposing soiled articles to more frequent exchanges of wash liquor, particularly, but not exclusively, when laundering multiple soiled articles 22 at once, or if one or more of soiled articles 22 is heavily soiled or stained.

Compartmentalization may also be achieved as shown, for example, in FIG. 7. Water permeable material 18 is inverted near the seam with water resistant material 16 to form funnel 30 with funnel opening 32. Funnel opening 32 may be narrowed relative to the seam between water permeable material 17 and water resistant material 16. Funnel opening 32 may be sized such that soiled absorbent articles 22 may pass easily through funnel opening 32 from the area defined by water resistant material 16 to the area defined by water permeable material 18 when second opening 14 is opened, as due to the operation of gravity or gentle shaking of bag 10. Funnel opening 32 may be sized such that soiled articles 22 will not pass easily through funnel opening 32 from the second compartment defined by water permeable material 18 back into the first compartment defined by water resistant material 16. Funnel 30 may alternatively be formed by a separate piece of water permeable material. Funnel 30 is not necessarily formed from the same material or material having the same structure (e.g., mesh or netting opening size or patterns) as water permeable material 18. Funnel opening 32 may be finished, perhaps with a separate edging material (not shown), to improve aesthetic appearance or durability. A one-way transfer of soiled articles may also be achieved via a flexible “tunnel” between the first and second compartments. Soiled articles 22 may move through the tunnel under the influence of gravity or purposeful manipulation of bag 10, with movement in the reverse direction unlikely without similar positioning or manipulation. A tunnel or funnel may be made of water resistant material 16, or water permeable material 18, or a material different from both water resistant material 16 and water permeable material 18. A one-way valve may be used with or in lieu of funnel 30.

FIGS. 8A and 8B show exemplary bags 10 having roll closures 28. Roll closure 28 is shown as a loop, however, roll closure 28 may comprise almost any securement means, including, but not limited to, magnets, ties, drawstrings, zippers, hook-and-loop, snaps, hook-and-eye, buttons with button holes, buttons with bands, and the like. Roll closure 28 may be elastic. FIG. 8A shows water permeable material 18 rolled inside water resistant material 16, with second opening 14 closed. Water resistant material 16, with water permeable material 18 cabined inside, may then be further rolled, and secured in a rolled condition using roll closure 28. This may be helpful in reducing the size of bag 10 for transportation or storage, and may help reduce the movement of soils within bag 10 prior to laundering. Lower mobility of soils within bag 10 may reduce the presence of soils near the seams and/or closures of bag 10, which may be more prone to leakage than water resistant material 16. For example, if water resistant material 16 is seamed using needle-made stitches, the holes in water resistant material 16 along the seam may be more prone to transmitting liquid soils than the rest of water resistant material 16. Of course, water resistant material 16 may be seamed using alternate joining technologies, such as ultrasonic welding, adhesive bonding, heat bonding, or other joining methods which may not increase the tendency to transmit liquid soils at the seam. The seams may also be formed using reinforced structures having folds of material or separate reinforcing strips that reduce the tendency to transmit liquid soils along puncture holes or other joining artifacts at the seam.

FIG. 8A shows water permeable material 18 cabined within a main compartment formed by water resistant material 16. Water permeable material 18 may also be cabined within water resistant material 16, but partially or wholly outside of the compartment defined by water resistant material 16. For example, FIG. 8B shows water permeable material 18 rolled against the outside of one wall of water permeable material 16.

Water permeable material 18 may be deployed prior to laundering bag 10. That is, in addition to opening second opening 14, water permeable material 18 may be unrolled or pulled out of the compartment defined by water resistant material 16. Water permeable material 18 may be deployed by opening second opening 14 and shaking or “snapping” bag 10 while holding bag 10 by water resistant material 16, such that soiled articles 22 are urged toward water permeable material 18, thereby encouraging the unfolding of water permeable material 18 from the compartment defined by water resistant material 16. In some embodiments, water permeable material 18 may not require deployment. In some embodiments, water permeable material 18 may be sized such that there is not much, or not any, material to deploy. That is, water permeable material 18 may span second opening 14, and might not be sized to fully surround one or more soiled articles 22.

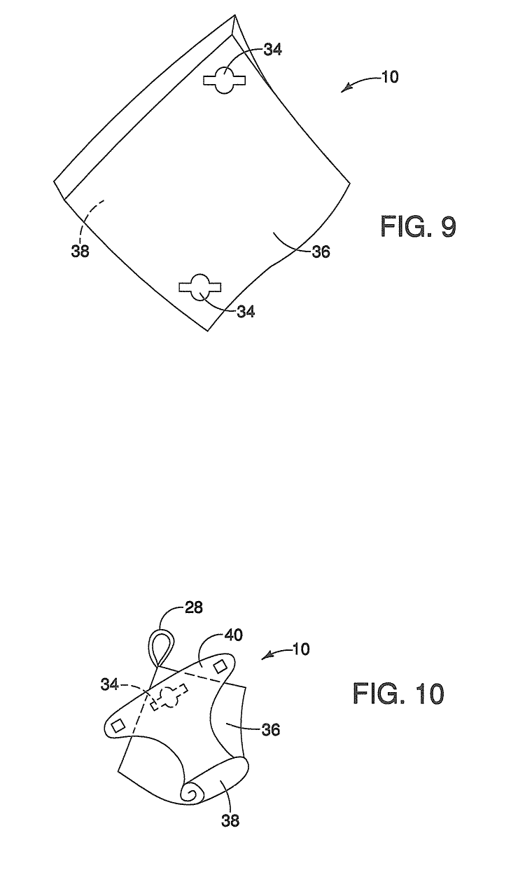

In some embodiments, bag 10 is configured to serve as a multipurpose surface. As shown in FIG. 9, bag 10 may have a first surface 36 and a second surface 38 on opposite exterior surfaces. First surface 36 and second surface 38 may be adapted such that bag 10 serves as a changing pad for changing an infant's diaper. For example, first surface 36 and second surface 38 may be smooth, water-resistant surfaces such that soils, from a diaper change or from potentially dirty surfaces where the diaper change is performed, such as a public restroom, can be easily wiped away from first surface 36 and second surface 38. In some embodiments, first surface 36 may be soft and/or absorbent, for the comfort of an infant placed on bag 10. For example, first surface 36 may comprise cotton knit, terry cloth, polyester knit, or any woven or nonwoven material providing a pleasant tactile sensation. If bag 10 is configured to be rolled, as shown in FIGS. 8A and 8B, first surface 36 may be disposed on the interior of the roll, such that soils absorbed by or disposed on first surface 36 will be sequestered when bag 10 is rolled. A soft and/or absorbent first surface 36 may alternatively be used as a burp cloth, a cleaning surface (as for wiping surfaces, such as highchairs, car seats, toilet seats, etc.), or a clean mat that a child may sit or lie on. Where soiled articles 22 are reusable outer covers used with an absorbent insert, the reusable covers may not be soiled regularly. That is, the reusable covers may not require laundering after every exudate release or diaper change. Thus, bag 10 may frequently be available for uses, such as use as a burp cloth, that might be unappealing when bag 10 contains soiled absorbent articles.

As shown in FIGS. 9 and 10, bag 10 may comprise connectors 34 for connecting bag 10 to an absorbent article cover 40. Connectors 34 may help hold absorbent article cover 40 in place while changing an absorbent article. For example, connectors 34 may help control loose, floppy structures of an absorbent article cover 40 while inserting an absorbent insert (not shown), or may help hold absorbent article cover 40 in place while applying the absorbent article to a wearer. An absorbent article cover 40, loaded with an absorbent insert, if used, may be placed against connectors 34 and rolled with bag 10, such that a ready-to-use absorbent article is pre-loaded and ready for application to a wearer if a previously worn absorbent article becomes soiled or otherwise unsuitable for further short-term (or long-term) use. This may facilitate quick changes by keeping necessary elements, such as a fresh outer cover and a fresh absorbent insert, close at hand.

Connectors 34 may be adapted to temporarily join absorbent article cover 40 to bag 10. For example, connectors 34 may comprise fasteners, including, but not limited to, snaps, hook-and-loop, ties, adhesives, cohesives, magnets, safety pins, and the like. Absorbent article cover 40 may comprise fasteners suitable for mating with connectors 34. For example, connectors 34 may comprise the hook portion of a hook-and-loop fastener, and absorbent article cover 40 may comprise the loop portion of a hook-and-loop fastener. Connectors 34 may not join absorbent article cover 40 to bag 10, but may reduce the movement of absorbent article cover 40 relative to bag 10 while they are in contact with one another. For example, connectors 34 may be areas of higher friction than the remainder of first surface 36, such that absorbent article cover 40 is less inclined to slip against the surface of connectors 34. For example, connectors 34 may have a static coefficient of friction of 0.5 or greater with regard to the material on the outside surface of absorbent article cover 40. Connectors 34 are shown in FIG. 9 as two discrete, shaped zones, however, connectors 34 may be of any size, shape, and quantity desired. In some embodiments, the entire first surface 36 of bag 10 may have a high coefficient of friction with regard to absorbent article cover 40. In some embodiments, no connectors 34 are present; however, an absorbent article cover 40 may still be rolled with bag 10 without connectors 34.

After fresh absorbent article cover 40 is applied to a wearer, the previously worn, now soiled, absorbent article 22 may be placed into bag 10. Bag 10 may then be rolled again. Bag 10 may provide a compact, soil-resistant solution for carrying, transporting, and laundering absorbent articles through an entire cycle of laundered-soiled-laundered. While bag 10 may be useful outside the home, it should be understood that bag 10 may also be used to store soiled articles 22 at the user's home or in an institutional setting, such as a daycare facility or nursing home. Providing a convenient and leak-resistant storage solution for soiled articles 22 may encourage laundering more than one soiled article 22 at a time, which may help to reduce the environmental impact of laundering reusable absorbent articles.

A kit may be provided for storing, transporting, or laundering reusable absorbent articles. The kit may comprise a bag 10 as described herein, and one or more reusable absorbent articles or reusable absorbent article components. For example, the kit may comprise one or more reusable outer covers. The kit may further comprise one or more absorbent inserts, which may be disposable or reusable. The bag and reusable absorbent article component(s) may be configured to temporarily or reversibly join the bag to one or more reusable absorbent article components. The bag may be of sufficient size to hold at least two of the reusable absorbent article components, or between 5 and 8 of the reusable absorbent article components, or up to 50 of the reusable absorbent article components. The kit may include directions for using the bag. The directions may explain how to temporarily or reversibly join the bag to one or more reusable absorbent articles, or how to convert the bag from a first configuration to a second configuration (as described above), or both. The directions may explain how to use the reusable absorbent articles or components. The directions may include text or informational images or both.

The kit may be intended to facilitate trial of an absorbent article which is at least partially reusable. For example, the kit may include one bag, one or two reusable outer covers, and between 5 and 10, or between 7 and 14, or between 6 and 18, absorbent inserts. The number of absorbent inserts in the kit may correspond, for example, to the average number of inserts used over a desired trial period. For example, a young infant may need a diaper change approximately 6-8 times per day. If it is desired that the young infant's caregiver try the reusable absorbent article for a trial period of 3 days, the kit may include between 18 and 24 disposable absorbent inserts, or at least 9 reusable absorbent inserts, or between 9 and 16 reusable absorbent inserts, to allow at least one full day's usage before it is necessary to launder the reusable absorbent inserts. Similarly, the number of reusable outer covers may correspond to the average number of soiled outer covers generated during the desired trial period.

The kit may be intended to facilitate regular use of an absorbent article which is at least partially reusable. For example, the kit may include two or three bags, seven or eight reusable outer covers, and at least 10, or at least 20, or at least 30, or at least 50 absorbent inserts. The kit may include reusable outer covers in more than one size. For example, if the absorbent article is adapted for an infant, the kit may include reusable outer covers in two or more sizes to accommodate anticipated physical growth as the infant matures. The kit may include reusable absorbent article components having different colors, designs, or patterns. The kit may include reusable absorbent article components having different functional properties. For example, the kit may include absorbent inserts with different absorptive capacities, such as a first absorptive capacity intended for day time use, when the insert may be changed after each exudate release or at relatively frequent intervals, and a second absorptive capacity intended for night time use, when the insert may be changed only after an extended period of use. As another example, the kit may include reusable outer covers having different styles or forms. For example, the kit may include a first reusable outer cover having a relatively large coverage area in the crotch and rear regions (near the wearer's perineum and back, respectively, during use) which may, for example, accommodate more modest users or provide improved leakage protection, and a second reusable outer cover having a relatively small coverage area in the crotch and front leg regions (near the wearer's perineum and front thighs, respectively, during use) which may, for example, provide improved comfort or fit for an active wearer.

### Test Methods

### Average Hole Size and Percent Open Area of Mesh

Average hole area (mm²) and percent open area of the holes (%) are measured by light microscopy with image analysis. A Nikon SMZ1500 microscope fitted with a 0.5× Nikon WD136 HR Plan Apo lens, and the stage illuminator is used. Magnification is set at 1.0× to give a viewing area of approximately 25×20 mm. Transmitted light images are acquired using an Evolution MP Color Digital Camera (Media Cybernetics, Bethesda, Md.) and processed using ImageJ software version 1.42 (NIH, Bethesda, Md.). Equivalent equipment may be used.

A 15 cm×15 cm test specimen is cut from the sample and prepared for analysis. Set the camera capture software to 8 bit mono. Turn the stage light on and place the specimen onto the microscope stage and focus the image. Remove the specimen and place a calibrated ruler on the stage. Take an image of the ruler for calibration of the images (top illumination is temporarily needed to image the ruler). The magnification and focus should not be changed after the calibration image is taken. Place the specimen back onto the stage and adjust the stage light until the holes are clearly illuminated. Collect five images at different positions along the surface of the specimen.

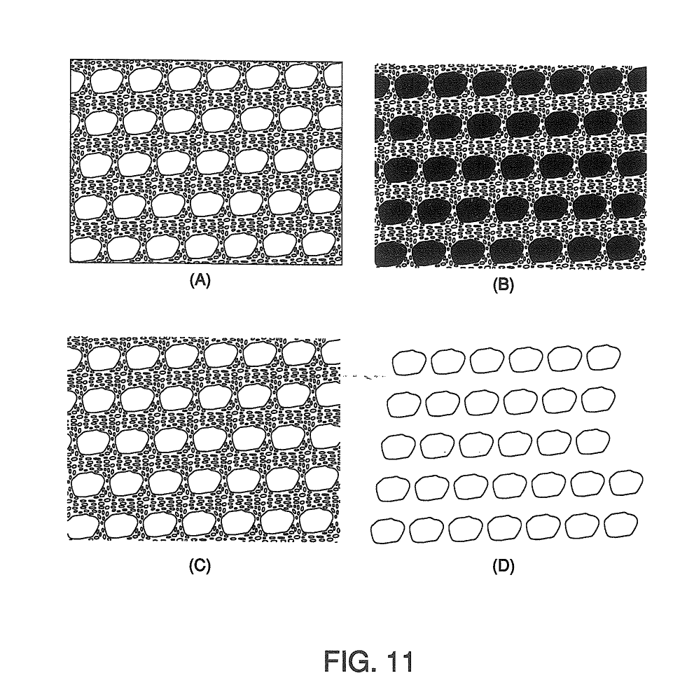

For illustration purposes only, FIG. 11 gives a mesh example at the different stages of image processing. Referring to FIG. 11, shown is the original image from the microscope camera (FIG. 11A), a threshold image in ImageJ (FIG. 11B), an image measuring total % open area from ImageJ (FIG. 11C), and an image measuring area of dominant holes from ImageJ (FIG. 11D).

Load the images from the microscope camera into ImageJ for analysis. Open the ruler image and calibrate the software for the number of pixels per mm of length. Open a specimen image and adjust the threshold by setting to default, black and white, with dark background. Set the software to include holes that are in contact with the edges of the image (for calculation of total open area), exclude any holes contained within the holes (fill holes), and exclude holes with an area less than 0.02 mm². Measure the total area of the holes and report as % Open Area to the nearest 0.1%.

Set the software to exclude holes that are in contact with the edges of the image (for calculation of average hole area), exclude any holes contained within the holes (fill holes), and exclude holes with an area less than 0.02 mm². Measure the hole areas. Examine output to determine if the hole area distribution is binary. An example of a binary distribution is given in FIG. 11C. If there are two distinct populations of holes, adjust the hole exclusion area so as to measure only the dominant (larger) hole population (FIG. 11D). Re-measure the hole areas and record the area of the holes to the nearest 0.01 mm².

This procedure is repeated with all five images of the specimen, and the individual results averaged. Report the averages as % Open Area to the nearest 0.1% and Hole Area to the nearest 0.01 mm²

### Hydrostatic Head Test

Hydrostatic head test is conducted as described in EDANA/INDA Worldwide Strategic Partners Method WSP 80.6 (50) using a TexTest FX 3000 Hydrostatic Head Tester, or equivalent. The test is performed using the 100 cm² test head, with a water temperature of 23±2° C. The test is modified to increase the pressure at a rate of 20 mbar/min. The test is terminated and pressure recorded after 3 separate drops are formed on the surface of the specimen. These drops must form in three different penetrating holes, and must originate at least 6 mm away from the edge of the clamp.

Using scissors, a specimen 14 cm by 14 cm is cut from the material to be tested. If the material, as it will be used, consists of multiple layers, the layers are tested as a composite. The side of the material which is intended to form the inside of the bag (i.e., the surface nearest the bag contents) is oriented toward the water for testing. Report the Hydrostatic head pressure to ±0.1 mbar.

### Modified AATCC Test Method 124-2001

In a wash cycle, a component of an absorbent article, such as an outer cover, is machine washed and machine dried according to the protocol from AATCC (American Association of Textile Chemists and Colorists) Test Method 124-2001, with the selected parameters and variations listed below.

a) Per section 6, Apparatus and materials, a Kenmore 600 (Heavy Duty—Super Capacity Plus—Quiet Pak) is used for the automatic washing machine, and a Maytag Commercial (such as model numbers MDE27MNACW, MDE15MNAYW, and MDE13MNACW) is used for the automatic tumble dryer.

b) Despite the instructions in Section 6, Apparatus and materials, the following ballast is used: Test Fabric style 493 from Testfabrics, Inc, West Pittston, Pa., which is cotton sheeting, with a thread count of 60×60, a weight of 151 gsm, and a size of 55′ by 39″.

c) Despite the instructions in Section 6, Apparatus and materials, the evaluation area is not configured according to section 6.7 and the apparatus of section 6.8 is not used. Instead, all visual evaluations are preformed under typical artificial lighting conditions (e.g. fluorescent light), which allows a person with normal vision to clearly see.

d) Despite the instructions in Section 7, Test Specimen, the component to be tested is (as necessary) entirely removed from the rest of the absorbent article, and (to the extent allowed by the removal) the component is tested as an undamaged whole. Up to three components of the same type are washed simultaneously.

e) Regarding the machine wash in Section 8.2.2, use the “large” setting on the machine for the water level, select a wash temperature of 32+/−3° C. (90+/−5° F.), and a rinse temperature of 16+/−3° C. (60+/−5° F.).

f) Regarding the settings in Section 8.2.2, select Normal/Cotton Sturdy, which has a washing time of 12 minutes, an initial spin time of 6 minutes, a refill time of 4 minutes, a rinse time of 5 minutes, and a final spin cycle time of 6 minutes.

g) Regarding the Drying in Section 8.3, select Cotton Sturdy and Whites & Colors.

h) Despite the instructions in Section 8.5, the steps of conditioning and preconditioning are not performed.

i) Despite the instructions in Section 9, Evaluation, these evaluation steps are not performed. Instead, the tested component is evaluated by one of skill in the art, to determine whether the testing has resulted in significant degradation to the appearance or performance of the article that would render it unsuitable for its intended functionality and/or use.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. The features illustrated or described in connection with one non-limiting embodiment may be combined with the features of other non-limiting embodiments. It is therefore intended to cover in the appended claims all such changes and modifications that are within the scope of this invention.

What is claimed is:

1. A bag comprising:
a water resistant material;
a water permeable material joined to the water resistant material to form a continuous interior space;
a first configuration, wherein the water permeable material is cabined within the water resistant material; and
a second configuration, wherein the water permeable material is exposed.

2. The bag of claim 1, further comprising a first opening to access the continuous interior space, and a second opening to expose the water permeable material.

3. The bag of claim 2, wherein the second opening comprises a closure having an open position and a closed position and which, in the closed position, holds the bag in the first configuration.

4. The bag of claim 1, further comprising a funnel between the water permeable material and the water resistant material within the continuous inner space.

5. The bag of claim 4, further comprising a one-way valve attached to the funnel.

6. The bag of claim 1, wherein the continuous interior space is resistant to liquid in the first configuration and permeable to liquid in the second configuration.

7. The bag of claim 1, further comprising at least one intercompartmental fastener between the water resistant material and the water permeable material.

8. The bag of claim 1, further comprising a closure to hold the bag in a rolled or folded configuration.

9. The bag of claim 1, wherein the bag has a first outer surface and a second outer surface opposite the first outer surface, at least one of the first outer surface and the second outer surface comprising a connector for holding an absorbent article.

10. The bag of claim 1, wherein the bag has a first outer surface and a second outer surface opposite the first outer surface, at least one of the first outer surface and the second outer surface comprising an absorbent material.

11. The bag of claim 1, wherein the water resistant material comprises a laminate of a water permeable material and a film.

12. The bag of claim 1, wherein the water permeable material comprises a mesh or netting.

13. The bag of claim 12, wherein the mesh or netting has an average hole size of at least 2 mm².

14. The bag of claim 13, wherein the mesh or netting has an average hole size of at least 5 mm².

15. A kit comprising a bag according to claim 1 and one or more reusable absorbent article components.

16. The kit of claim 15, wherein the bag and at least one of the one or more reusable absorbent article components are configured to be reversibly joined together.

17. The kit of claim 15, further comprising instructions for using the bag.

18. The kit of claim 17, further comprising instructions for using the one or more reusable absorbent article components.

19. The kit of claim 15, wherein the one or more reusable absorbent article components comprise a reusable outer cover.

20. The kit of claim 19, further comprising at least one disposable absorbent insert.
